(19) United States
(12) Patent Application Publication
(10) Pub. No.: US 2025/0230223 A1
(43) Pub. Date: Jul. 17, 2025

(54) A NOVEL H1N1 ANTIBODY

(71) Applicant: UNIVERSITY OF GEORGIA RESEARCH FOUNDATION, INC., Athens, GA (US)

(72) Inventors: Ralph A. TRIPP, Athens, GA (US); Leslie Patrick JONES, Athens, GA (US)

(21) Appl. No.: 18/849,109

(22) PCT Filed: Mar. 21, 2023
(86) PCT No.: PCT/US2023/064782

§ 371 Date: Sep. 20, 2024

Related U.S. Application Data
(60) Provisional application No. 63/322,047, filed on Mar. 21, 2022.

(51) Int. Cl. C07K 16/10; A61K 39/00; A61P 37/04

(52) U.S. Cl. CPC C07K 16/1018; A61P 37/04; A61K 2039/505; C07K 2317/33; C07K 2317/76

(57) ABSTRACT

Disclosed are novel H1N1 antigen binding molecules and methods of their use for the treatment and/or prevention of influenza A virus.

This application claims the benefit of U.S. Provisional Application No. 63/322,047, filed on Mar. 21, 2022, which is incorporated herein by reference in its entirety.

### I. BACKGROUND

Each year, seasonal influenza causes over 300,000 hospitalizations and 36,000 deaths in the U.S. alone (Simonsen et al., Lancet Infect Dis 7:658-66, 2007). The emergence of the novel H1N1 influenza virus in 2009 demonstrated how quickly a new influenza pandemic can sweep across the world. There are currently two influenza vaccine approaches licensed in the United States—the inactivated, split vaccine and the live-attenuated virus vaccine. The inactivated vaccines can efficiently induce humoral immune responses, but generally only poor cellular immune responses. Live virus vaccines cannot be administered to immunocompromised or pregnant patients due to their increased risk of infection. Because the influenza virus rapidly changes, a new version of the vaccine is developed twice a year to account for the southern hemisphere and northern hemisphere influenza season. Despite these considerable efforts, seasonal influenza remains a significant problem for our healthcare system. What are needed are new Influenza A vaccines and treatment methods.

### II. SUMMARY

Disclosed are methods and compositions related to influenza H1N1 binding molecules.

In one aspect, disclosed herein are influenza A antigen (including, but not limited to, for example H1N1 antigen) binding molecules (such as, for example a monoclonal antibody, polyclonal antibody, immunotoxin, single chain variable domain, nanobody, bi-specific antibody (including, but not limited to a a BiTE, Cross BiTE, and/or Tand BiTE), tri-specific antibody, chimeric antigen receptor (CAR) T cell, CAR natural killer (NK) cell, or CAR macrophage (CARMA)) comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively (such as, for example SEQ ID NO: 4) and/or a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively (such as, for example SEQ ID NO: 8). In some aspects, disclosed herein are influenza A antigen binding molecules of any preceding aspect (including, but not limited to, for example H1N1 antigen binding molecules), further comprising a constant domain as set forth in SEQ ID NO: 9. In some aspect, the H1N1 antigen binding molecule binds the hemagglutinin (HA) protein of a influenza virus.

Also disclosed herein are methods of treating, inhibiting, decreasing, reducing, ameliorating, and/or preventing an influenza A virus infection (such as, for example a swine influenza virus (SIV) infection including, but not limited to H1N1, H1N2, H2N1, H3N1, H3N2, and H2N2 infection) in a subject comprising administering to the subject the influenza A antigen (including, but not limited to, for example H1N1 antigen and/or HA antigen) binding molecules (such as, for example a monoclonal antibody, polyclonal antibody, immunotoxin, single chain variable domain, nanobody, bi-specific antibody (including, but not limited to a a BiTE, Cross BiTE, and/or Tand BiTE), tri-specific antibody, chimeric antigen receptor (CAR) T cell, CAR natural killer (NK) cell, or CAR macrophage (CARMA)) of any preceding aspect (including, but not limited to a H1N1 antigen binding molecule). For example, disclosed herein are methods of treating, inhibiting, decreasing, reducing, ameliorating, and/or preventing in influenza virus infection in a subject comprising administering to the subject a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively (such as, for example SEQ ID NO: 4) and/or a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively (such as, for example SEQ ID NO: 8). In some aspects, disclosed herein are H1N1 antigen binding molecules of any preceding aspect, further comprising a constant domain as set forth in SEQ ID NO: 9. The influenza A antigen binding molecule can be administered prophylactically or therapeutically. Thus, in one aspect, the influenza A antigen binding molecule can be administered before our after exposure to the Influenza A virus or the onset of symptoms.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show HPLC analysis and SDS-PAGE analysis of H1N1 antibody.

FIGS. 2A and 2B show the detection of the antibody compared to controls. FIG. 2A shows primary antibody detection. FIG. 2B shows the detection and ability of the antibody to bind 10 ug of A/SW/NEB A/101444614/2013 lysate (2.7×10⁶ pfu/ml) coated on an EIA plate.

FIG. 3 shows a HAI assay for A/Swine/Nebraska 2013(H1N1) vs recombinant αH1N1 Antibody.

FIGS. 4A, 4B, and 4C show the therapeutic Effect in Pigs Infected with Swine Influenza Virus (SIV) A/Swine/Nebraska 2013 (H1N1). FIG. 4A shows the mean QPCR in pigs following viral exposure. FIG. 4B shows the mean TCID50 in pigs following viral exposure. FIG. 4C shows the mean weight of the pigs following viral exposure.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-H1N1 Influenza hemagglutinin (HA) antigen binding molecule is disclosed and discussed and a number of modifications that can be made to a number of molecules including the anti-H1N1 Influenza hemagglutinin (HA) antigen binding molecule are discussed, specifically contemplated is each and every combination and permutation of the anti-H1N1 Influenza hemagglutinin (HA) antigen binding molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are H1N1 antigen binding molecules comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively (such as, for example SEQ ID NO: 4) and/or a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively (such as, for example SEQ ID NO: 8). In some aspects, disclosed herein are H1N1 antigen binding molecules, further comprising a constant domain as set forth in SEQ ID NO: 9. In some aspect, the H1N1 antigen binding molecule binds the hemagglutinin (HA) protein of a influenza virus.

It is understood and herein contemplated that the influenza A antigen (including, but not limited to, for example H1N1 antigen) binding molecules can comprise any antigen binding molecule known in the art, including, but not limited to a monoclonal antibody, polyclonal antibody, immunotoxin, single chain variable domain, nanobody, bi-specific antibody (including, but not limited to a a BiTE, Cross BiTE, and/or Tand BiTE), tri-specific antibody, chimeric antigen receptor (CAR) T cell, CAR natural killer (NK) cell, or CAR macrophage (CARMA) or any functional fragment thereof.

### 1. Antibodies

### (1) Antibodies Generally

The term “antibodies” is used herein in abroad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with H1N1 HA. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, VHH, and the like, including hybrid fragments. Thus, fragments of the antibodies that retain the ability to bind their specific antigens are provided. For example, fragments of antibodies which maintain H1N1 HA binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti-H1N1 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Immunotoxin

It is understood and herein contemplated that the disclosed H1N1 binding molecules can be an immunotoxin. By “immunotoxin”, it is meant a chimeric protein made of an antibody or modified antibody or antibody fragment (also called in the present application “antibody”), covalently linked to a fragment of a toxin. For use with the disclosed immunotoxins, the toxin can be diphtheria toxin, Pseudomonas exotoxin, A chain of ricin, ribosome inactivating proteins gelonin, saporin, bouganin, pokeweed antiviral protein, and dodecandron. Thus in one aspect, disclosed herein are H1N1 antigen binding molecules comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively (such as, for example SEQ ID NO: 4) and/or a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively (such as, for example SEQ ID NO: 8) further comprising a toxin (such as, for example, diphtheria toxin, Pseudomonas exotoxin, A chain of ricin, ribosome inactivating proteins gelonin, saporin, bouganin, pokeweed antiviral protein, and dodecandron); wherein the toxin is covalently attached to the H1N1 binding molecule (such molecules, being referred to as an H1N1 immunotoxin). In some aspects, disclosed herein are H1N1 immunotoxins, further comprising a constant domain as set forth in SEQ ID NO: 9. In some aspect, the H1N1 antigen binding molecule binds the hemagglutinin (HA) protein of a influenza virus.

### 3. Chimeric Antigen Receptor Cell

Chimeric antigen receptor (CAR) cells (such as, for example, a CAR T cell, CAR macrophage (CARMA), CAR natural killer (NK) cell, or CAR NK T) are immune cells (such as, for example T cells, macrophage, NK cells, or NK T cells) that have been modified to express a chimeric antigen receptor that targets a peptide, protein, or ligand on the surface of a target cell thereby directing the modified immune cell to the target. The extracellular domain of a typical CAR consists of the VH and VL domains-single-chain fragment variable (scFv)—from the antigen binding sites of a monoclonal antibody. The scFv is linked to a flexible transmembrane domain followed by a tyrosine-based activation motif such as that from CD3ζ. Second and third generation CARs include additional activation domains from co-stimulatory molecules such as CD27, CD28, inducible T cell co-stimulator (ICOS), OX40, and/or CD137 (41BB) which serve to enhance T cell survival and proliferation. In one aspect, disclosed herein CAR T cells, CAR macrophage, CAR NK cells, and CAR NK T cells; wherein the CAR comprises an H1N1 binding molecule comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively (such as, for example SEQ ID NO: 4) and/or a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively (such as, for example SEQ ID NO: 8). In some aspects, the H1N1 binding molecule of the CAR immune cell (i.e., the chimeric antigen receptor itself) is linked to a flexible transmembrane domain followed by a tyrosine-based activation motif such as that from CD3□. In some aspects, the CAR further comprises CD27, CD28, inducible T cell co-stimulator (ICOS), OX40, and/or CD137 (41BB) co-stimulatory domains. In some aspects, the CAR can further comprises an IL-12 inducer and/or an IL-2 receptor β chain.

### 4. Homology/Identity

It is understood that one way to define any known variants and derivatives or those that might arise, of the disclosed genes and proteins herein is through defining the variants and derivatives in terms of homology to specific known sequences. For example, SEQ ID Nos: 1-9 set forth particular sequences of CDRs and heavy and light chain variable domain for an anti-H1N1 Influenza hemagglutinin (HA) antigen binding molecule. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 5. Peptides

### a) Protein Variants

As discussed herein there are numerous variants of the anti-H1N1 Influenza hemagglutinin (HA) antigen binding molecule. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NOs: 1-4 set forth a particular sequence of complementarity determining regions (CDRs) and heavy chain of an anti-Influenza A virus H1N1 antigen binding molecule and SEQ ID NOs: 5-8 set forth a particular sequence of complementarity determining regions (CDRs) and light chain of an anti-Influenza A virus H1N1 antigen binding molecule. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular anti-H1N1 Influenza hemagglutinin (HA) antigen binding molecules or CDRs from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Treating Influenza

It is understood and herein contemplated that the disclosed Influenza A antigen binding molecules (including, but not limited to H1N1, H1N2, H2N1, H3N1, H3N2, and H2N2 binding molecules) can be used in the treatment, inhibition, reduction, amelioration, and/or prevention of a influenza A virus infection (such as, for example a swine influenza virus (SIV)). Accordingly, disclosed herein are methods of treating, inhibiting, decreasing, reducing, ameliorating, and/or preventing an influenza A virus infection (such as, for example a swine influenza virus (SIV) infection including, but not limited to H1N1, H1N2, H2N1, H3N1, H3N2, and H2N2 infection) in a subject comprising administering to the subject any of the Influenza A antigen binding molecules (such as, for example a H1N1 antigen binding molecule) disclosed herein. For example, disclosed herein are methods of treating, inhibiting, decreasing, reducing, ameliorating, and/or preventing in influenza A virus infection in a subject comprising administering to the subject a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively (such as, for example SEQ ID NO: 4) and/or a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively (such as, for example SEQ ID NO: 8). In some aspects, disclosed herein are H1N1 antigen binding molecules, further comprising a constant domain as set forth in SEQ ID NO: 9.

The influenza A antigen binding molecule can be administered prophylactically or therapeutically. Thus, in one aspect, the influenza A antigen binding molecule can be administered before or after exposure to the Influenza A virus or the onset of symptoms. In one aspect, the Influenza A antigen binding molecule is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 42, 45, 48, 56, 58, 59, 60, 61, 62, 90 days, 3, 4, 5, 6, 7, 8, 9, 10, 11, 12, 13, 14, 15, 16, 17, 18, 19, 20, 21, 22, 23, 24 months, 3, 4, 5, 6, 7, 8, 9, 10 or more years prior to exposure to influenza A virus. Also disclosed herein are methods of treating an influenza A infection, wherein the influenza A binding molecule is administered 1, 2, 3, 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14, 15, 16, 17, 18 days after exposure to influenza virus or the onset of symptoms.

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

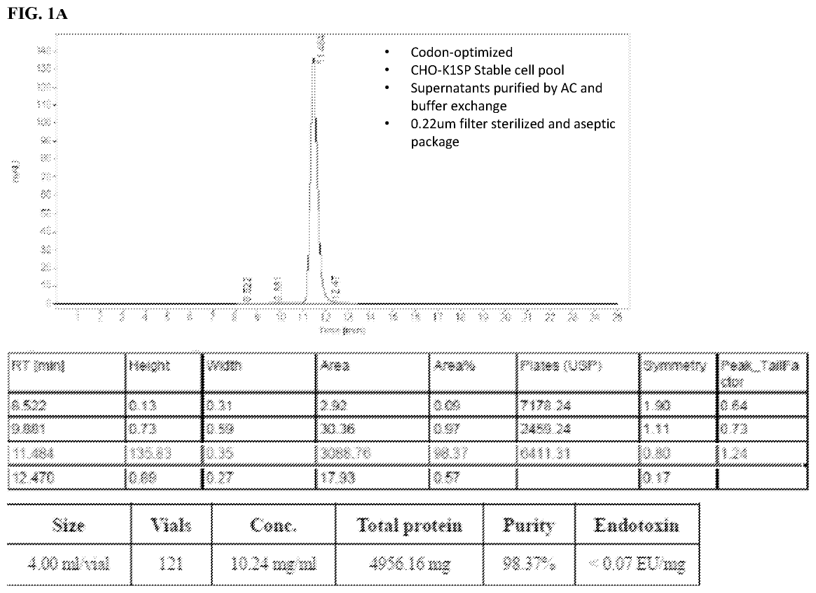

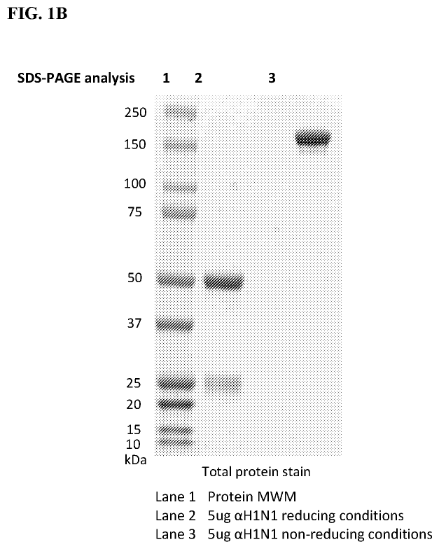

We designed and produced a human H1N1 IgG1 antibody (raH1N1). the antibody (Ab) was produced at Genscript (Piscataway, NJ) labs and expressed in a stably-transfected CHO cell line. The the Ab was expressed at 2 g/L of culture in a fed-batch process. Recombinant Ab was purified from culture supernatant over a Protein A sepharose column in PBS. Preparation was confirmed mycoplasm-free by PCR analysis. As shown in FIGS. 1A and 1B, Antibody production was successfully performed in CHO-KISP stable cell culture. Target protein antibody was detected with molecular mass of 150 kDa by SDS-PAGE analysis. Purity of the target protein was 98.37% determined by SEC-HPLC. A total of 4.95 g target protein (10.24 mg/ml, endotoxin <0.07 EU/mg). Testing consisted of SDS-PAGE analysis to confirm correct heavy and light chain bands.

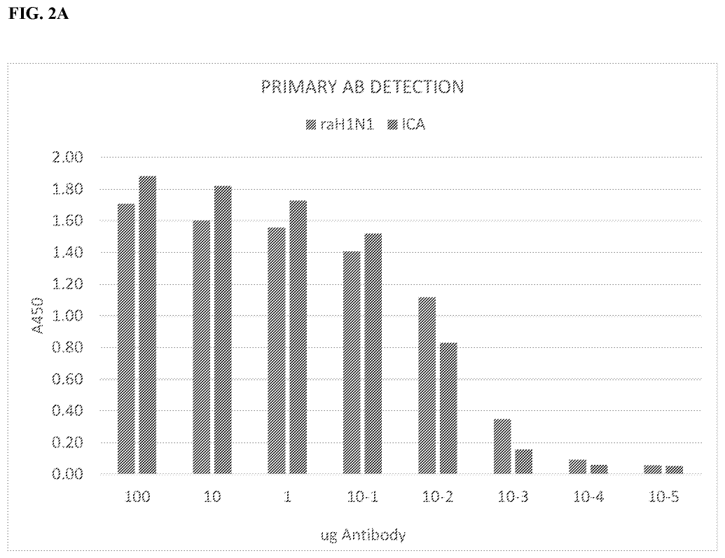

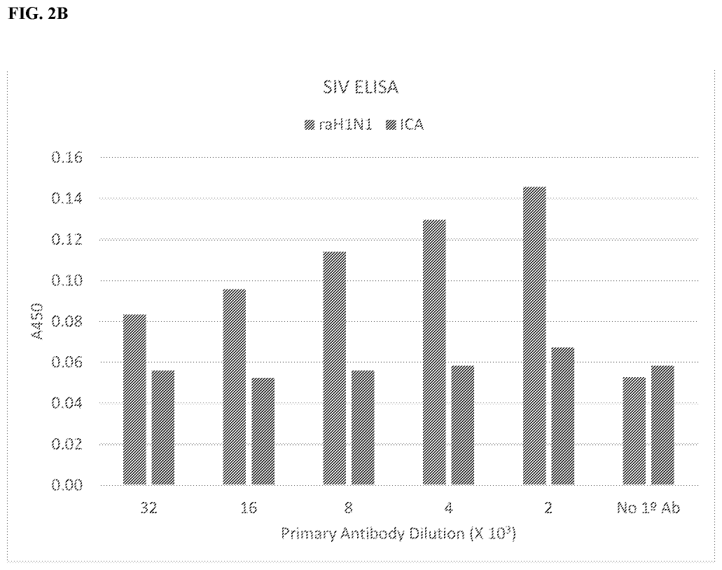

Having produced an antibody that bound H1N1, we wanted to characterize the antibody and its binding specificity. Compared to an isotype control, the H1N1 antibody had a similar mass to an isotype control antibody (ICA) as measured by ELISA (FIG. 2A). We also looked at the specificity of the raH1N1 antibody. ELISA plates were coated with 10 ug of A/SW/NEB A/101444614/2013 lysate (2.7×10⁶ pfu/ml) and the binding to the antigen was assayed. As shown in FIG. 2B, raH1N1 bound to the target in as little as a 1:32×10³ dilution with increasing signal as the dilution decreased whereas the ICA never produced a signal above background. Binding activity was confirmed by ELISA against both pandemic H1N1 virus and swine H1N1 virus (SIV)

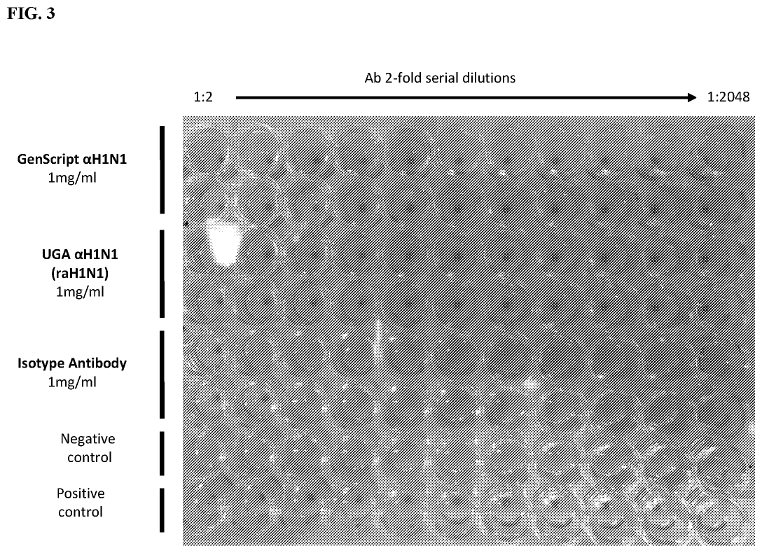

We next tested the ability of the raH1N1 antibody to neutralize swine H1N1 virus (SIV) (FIG. 3). 105 PFU of Influenza A/Swine/Nebraska 2013 were coated onto microwell plates and a raH1N1 antibody, a GenScript anti-H1N1 antibody, an isotype control, a positive control or a negative control at the indicate dilutions. The mixture was incubated for 1 hour after which TPCK trypsin was added and then transferred to washed MDCK cells for a 72 hours incubation. 50 ul was transferred to round bottom plates and 50 ul of 0.05% Turkey red blood cells were added. Results show that at all dilutions tested, the raH1N1 antibody was able to neutralize swine H1N1 virus.

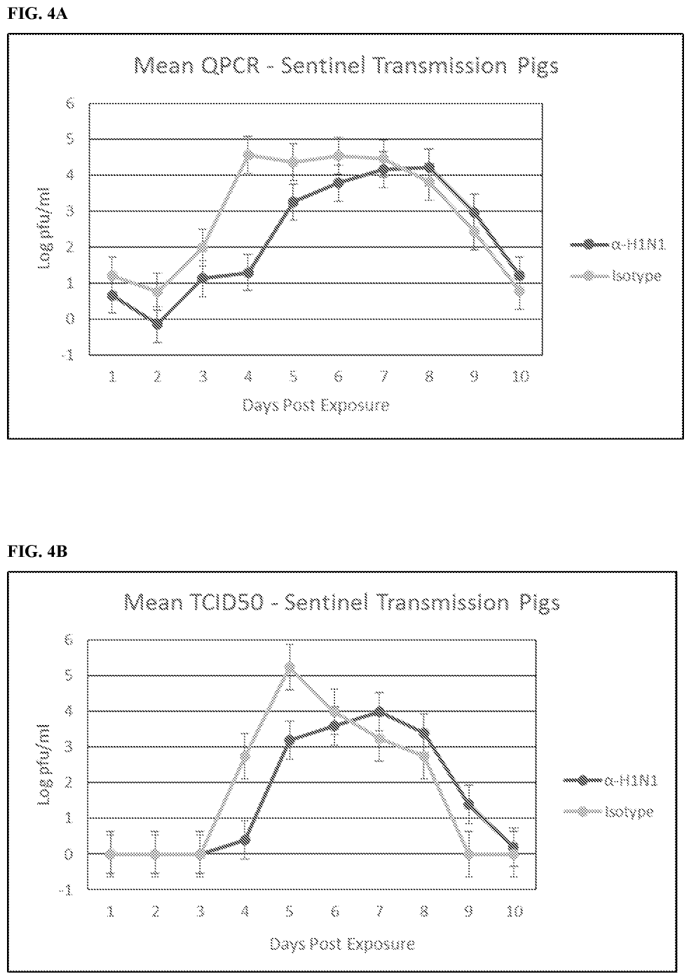

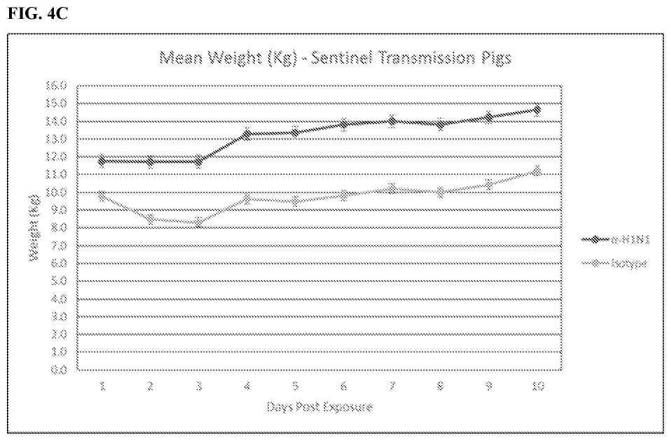

Next we examined the therapeutic effect of the raH1N1 antibody in pigs infected with Swine Influenza Virus (Se) A/Swine/Nebraska 2013 (H1N1) (FIG. 4). As measured by qPCR, α-H1N1 treatment of pigs lowers magnitude of infection by 3 logs and delays peak of transmitted virus (day 4 vs day 7). This finding was confirmed with tissue culture infectious dose (TCID50) which showed that the magnitude of infection was lowered in α-H1N1 treated pigs by 2 Logs and the antibody delays peak of transmitted virus (day 5 vs day 7) in agreement with qPCR data. Looking at the weight of infected animals, α-H1N1 treatment of pigs increases the end-point weights by >3 Kg vs Isotype Control antibody treatment and eliminates early weight loss. Additionally, the α-H1N1 antibody reduced virus load transmitted to naïve pigs, and prolonged the time to peak virus titers in the lungs; and prevented weight loss and disease.

E. Sequences
SEQ ID NO: 1 H1N1 antibody Heavy chain CDR1
SNYYWG
SEQ ID NO: 2 H1N1 antibody Heavy chain CDR2
SIYHSGSTYYKPSLES
SEQ ID NO: 3 H1N1 antibody Heavy chain CDR3
HVRSGYPDTAYYFDK
SEQ ID NO: 4 Heavy chain with human IgG1 C region:
METDTLLLWVLLLWVPGSTPGEVQLVESGPGLVKPSDILSLTCAV
SGYSISSNYYWGWIRQPPGKGLEWIGSIYHSGSTYYKPSLESRLG
ISVDTSKNQFSLKLSFVSAADTAVYYCARHVRSGYPDTAYYFDKW
GQGTLVTVSS
SEQ ID NO: 5 H1N1 antibody light chain CDR1
GGNNIGTKVLH
SEQ ID NO: 6 H1N1 antibody light chain CDR2
DDSDRPS
SEQ ID NO: 7 H1N1 antibody light chain CDR3
QVWDISTDQAV
SEQ ID NO: 8 Light chain (Lambda-2):
MAWALLLLGLLSHCTGSVTSYVLTQPPSVSVAPGETARISCGGNN
IGTKVLHWYQQTPGQAPVLVVYDDSDRPSGIPERFSGSNSGNTAT
LTISRVEVGDEADYYCQVWDISTDQAVFGGGTKLTVLGQPKAAPS
VTLFPPSSEELQANKATLVCLISDFYPGAVTVAWKADSSPVKAGV
ETTTPSKQSNNKYAASSYLSLTPEQWKSHRSYSCQVTHEGSTVEK
TVAPTECS
SEQ ID NO: 9: Heavy Chain Constant Domain
AGTEGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

1. An Influenza A antigen binding molecule comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively.

2. The Influenza A antigen binding molecule of claim 1, wherein the H1N1 antigen binding molecule further comprises a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively.

3. The Influenza A antigen binding molecule of claim 1, wherein the heavy chain comprises the sequence as set forth in SEQ ID NO: 4.

4. The Influenza A antigen binding molecule of claim 2, wherein the light chain comprises the sequence as set forth in SEQ ID NO: 8.

5. (canceled)

6. The Influenza A antigen binding molecule of claim 1, further comprising a constant domain as set forth in SEQ ID NO: 9.

7. The Influenza A antigen binding molecule of claim 1, wherein the antigen binding molecule comprises a monoclonal antibody, polyclonal antibody, immunotoxin, single chain variable domain, nanobody, bi-specific antibody, tri-specific antibody, chimeric antigen receptor (CAR) T cell, CAR natural killer (NK) cell, or CAR macrophage (CARMA).

8. A method of treating an influenza A viral infection in a subject comprising administering to the subject the Influenza A antigen binding molecule of claim 1.

9. A method of treating an influenza A viral infection in a subject comprising administering to the subject an Influenza A antigen binding molecule comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively.

10. The method of treating an influenza A viral infection of claim 9, wherein the influenza A antigen binding molecule further comprises a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively.

11. The method of treating an influenza viral infection of claim 9, wherein the heavy chain comprises the sequence as set forth in SEQ ID NO: 4.

12. The method of treating an influenza viral infection of claim 9, wherein the light chain comprises the sequence as set forth in SEQ ID NO: 8.

13. The method of preventing or inhibiting an influenza A viral infection in a subject comprising administering to the subject the influenza A antigen binding molecule of claim 1.

14. A method of preventing or inhibiting an influenza A viral infection in a subject comprising administering to the subject an Influenza A antigen binding molecule comprising a heavy chain variable region; wherein the heavy chain variable region comprises three complementarity determining regions (CDRs) referred to as CDR1, CDR2, and CDR3 as set forth in SEQ ID NO: 1, SEQ ID NO: 2, and SEQ ID NO: 3, respectively.

15. The method of preventing or inhibiting an influenza viral infection of claim 14, wherein the influenza A antigen binding molecule further comprises a light chain variable region and wherein the light chain variable region comprises three complementarity determining regions (CDRs) referred to as CDR1, CDR2, and CDR3 as set forth in SEQ ID NO: 5, SEQ ID NO: 6, and SEQ ID NO: 7, respectively.

16. The method of preventing or inhibiting an influenza viral infection of claim 14, wherein the heavy chain comprises the sequence as set forth in SEQ ID NO: 4.

17. The method of preventing or inhibiting an influenza viral infection claim 14, wherein the light chain comprises the sequence as set forth in SEQ ID NO: 8.

18. The method of treating an influenza virus infection of claim 7; wherein the influenza virus is a swine influenza virus.

19. The method of treating an influenza virus infection of claim 18 or preventing or inhibiting an influenza viral infection of claim 18; wherein the influenza virus is an H1N1 influenza virus.

20. The method of preventing or inhibiting an influenza viral infection of any of claim 14; wherein the influenza virus is a swine influenza virus.

21. The method of treating an influenza virus infection of claim 18 or preventing or inhibiting an influenza viral infection of claim 20; wherein the influenza virus is an H1N1 influenza virus.


SEQUENCE LISTING (9 sequences)

SEQ ID NO: 1 - protein, 6 aa, synthetic construct
SNYYWG

SEQ ID NO: 2 - protein, 16 aa, synthetic construct
SIYHSGSTYYKPSLES

SEQ ID NO: 3 - protein, 15 aa, synthetic construct
HVRSGYPDTAYYFDK

SEQ ID NO: 4 - protein, 145 aa, synthetic construct
METDTLLLWVLLLWVPGSTPGEVQLVESGPGLVKPSDILSLTCAVSGYSISSNYYWGWIRQPPGKGLEWIGSIYHSGSTYYKPSLESRLGISVDTSKNQFSLKLSFVSAADTAVYYCARHVRSGYPDTAYYFDKWGQGTLVTVSS

SEQ ID NO: 5 - protein, 11 aa, synthetic construct
GGNNIGTKVLH

SEQ ID NO: 6 - protein, 7 aa, synthetic construct
DDSDRPS

SEQ ID NO: 7 - protein, 11 aa, synthetic construct
QVWDISTDQAV

SEQ ID NO: 8 - protein, 233 aa, synthetic construct [sequence omitted]

SEQ ID NO: 9 - protein, 329 aa, synthetic construct
AGTEGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG